(19) United States
(12) Patent Application Publication
(10) Pub. No.: US 2007/0015799 A1
(43) Pub. Date: Jan. 18, 2007

(54) Glyoxalase inhibitors

(76) Inventors: Mark Ashton, Abingdon Oxfordshire (GB); Alan Davidson, Abingdon, Oxfordshire (GB); Russell Thomas, Oxfordshire (GB); Mark Whittaker, Oxfordshire (GB)

Correspondence Address: NIXON & VANDERHYE, PC, 901 NORTH GLEBE ROAD, 11TH FLOOR, ARLINGTON, VA, 22203, US

(21) Appl. No.: 10/556,901

(22) PCT Filed: May 14, 2004
(86) PCT No.: PCT/GB04/02101

§ 371 Date: Feb. 2, 2006

(30) Foreign Application Priority Data
May 15, 2003 (GB) 0311195.2

(51) Int. Cl. A61K 31/4436; A61K 31/44; A61K 31/381

(52) U.S. Cl. 514/342; 514/357; 514/447; 514/616; 546/280.4; 546/336; 549/76; 564/152

(57) ABSTRACT

This invention relates to compounds of formula (I) which are glyoxalase I inhibitors, pharmaceutical salts or compositions comprising such compounds, and the use of such compositions and compounds to treat various conditions alleviated by the inhibition of glyoxalase 1. Wherein X is N or CH. R² is H, CF₃; or optionally substituted C₅₋₆ aryl, C₃₋₇ cycloalkyl, C₅₋₇ heterocyclyl. R³ is H; or optionally substituted C₅₋₆ aryl, C₃₋₇ cycloalkyl, C₅₋₇ heterocyclyl. Alternatively R² and R³ together form an optionally substituted C₃₋₄alkylene group wherein L³ and L⁴ are single bonds thus forming a C₅₋₆ ring fused with the aromatic ring to which L³ and L⁴ are attached. L³ and L⁴ are independently selected from a single bond, optionally substituted C₁₋₄ alkylene, -L⁹YN(OH)C(═O)L¹⁰- and -L⁹C(═O)N(OH)YL¹⁰-, wherein L⁹ and L¹⁰ are independently selected from optionally substituted C₁₋₄ alkylene, C₅₋₆ arylene, C₁₋₄ alkylene-C₅₋₆arylene and a single bond, wherein Y is NH or a single bond.

This invention relates to compounds which are glyoxalase I inhibitors, pharmaceutical compositions comprising such compounds, and the use of such compositions and compounds to treat various conditions alleviated by the inhibition of glyoxalase I.

### BACKGROUND OF THE INVENTION

Methyl glyoxal (MG) is an endogenous cytotoxic agent that is formed in cells as a consequence of glycolysis. The glyoxalase system converts 2-oxoaldehydes such as MG into the corresponding 2-hydroxy acid in two consecutive steps. MG is converted to D-lactate via the intermediate S-D-lactoylglutathione. The glyoxalase system comprises two enzymes, glyoxalase I and glyoxalase II. Glyoxalase I is the rate limiting enzyme, and catalyses the formation of S-D-lactoylglutathione from the hemithioacetal formed non-enzymatically from MG and reduced glutathione (GSH). Glyoxalase II catalyses the hydrolysis of S-D-lactoylglutathione to D-lactate, reforming the GSH consumed in the glyoxalase I-catalysed reaction (Thornalley et al., Crit. Rev. Oncol. Haematol. 20, 99 (1995)).

High levels of MG form DNA adducts and are generally toxic to cells. Cells having high glycolytic rates such as tumour cells and certain parasites have increased levels of glyoxalase I, which is believed to be the major detoxification pathway for MG. Glyoxalase-I levels were shown to be higher in 38 human cancer cell lines than in normal cells (Sakamoto et al., Clin. Cancer Res. 7, 2513 (2001)). Elevated glyoxalase I levels were observed in the following human cancer types: lung (Sakamoto et al., ibid.), prostate (Sakamoto et al., ibid.; Davidson et al., J. Urol. 161, 690 (1999); Samadi et al., Urology 57, 183 (2001)), colon (Ranganathan et al., Biochim. Biophys. Acta 1182, 311 (1993)), leukemia (Sakamoto et al., Blood 95, 3214 (2000)) and breast (Rulli et al., Breast Canc. Res. Treat 66, 67 (2001)). Agents that lead to an accumulation of MG, such as glyoxalase inhibitors, might be expected to exert an anti-tumor action (Thornalley et al., Gen. Pharmacol. 27, 565 (1996)) and are therefore likely to have a beneficial effect on patients suffering from various forms of cancer.

Prototype peptidic glyoxalase I inhibitors have been synthesised, based on knowledge of the substrate of glyoxalase I, i.e. the hemithioacetal formed from MG and GSH (Johansson et al., Mol. Pharmacol. 57, 619 (2000); Thornalley et al. J. Med. Chem. 39, 3409 (1996); Kalsi et al., J. Med. Chem. 42, 3981 (2000); Sharkey et al., Cancer Chemother. Pharmacol. 46, 156 (2000)). Such inhibitors have been shown to acutely increase MG levels and induce apoptosis in cancer cells. Additionally they have been shown to exert anti-cancer effects in vivo, both on their own and synergising with existing cytotoxic agents (Thornalley et al., Biochem. Pharmacol. 51, 1365 (1996); Sakamoto et al., Blood 95, 3214 (2000); Sharkey et al., ibid.). Moreover, there is increasing evidence that tumour cell resistance to certain cytotoxics (adriamycin, etoposide) may result, in part, from the over-activity of glyoxalase I (Sakamoto et al., Blood 95, 3214 (2000); Johansson et al., ibid.).

Known glyoxalase I inhibitor compounds are generally peptidic and require esterification to gain access to the interior of the cell where glyoxalase I is found. It is therefore desirable to find classes of glyoxalase I inhibitor compounds which are non-peptidic and hence have greater potential as therapeutic agents.

U.S. Pat. No. 4,898,870 describes pyrroloquinoline quinone compounds in relation to glyoxalase I inhibition, although no activity data for glyoxalase I inhibition is disclosed. WO 99/35128 is related to competitive inhibitor compounds of glyoxalase I, and a method of generating such inhibitors inside tumour cells using an acyl-interchange reaction between a membrane-permeable prodrug and intracellular glutathione.

The invention provides further classes of glyoxalase I inhibitor compounds which are non-peptidic, and therefore have greater potential as therapeutic agents.

### SUMMARY OF THE INVENTION

A first aspect of the present invention provides a compound of formula I: 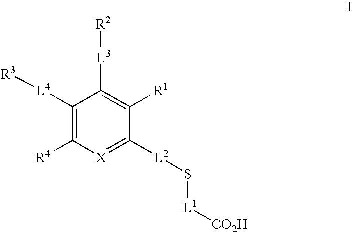
wherein

X is N or CH;

R¹ is H, cyano, halo, hydroxy, hydroxamic acid, sulfhydryl or —NH₂; or C₁₋₄ alkyl optionally substituted by cyano, halo, hydroxy, hydroxamic acid, sulfhydryl or —NH₂; or —OR, —NHR, —NR₂ or —SR wherein R is C₁₋₄ alkyl optionally substituted by cyano, halo, hydroxy, hydroxamic acid, sulfhydryl or —NH₂;

R² is H, CF₃; or optionally substituted C₅₋₆ aryl, C₃₋₇ cycloalkyl, C₅₋₇ heterocyclyl or together with R³ an optionally substituted C₃₋₄ alkylene group wherein L³ and L⁴ are single bonds thus forming a C₅₋₆ ring fused with the aromatic ring to which L³ and L⁴ are attached;

R³ is H; or optionally substituted C₅₋₆ aryl, C₃₋₇ cycloalkyl, C₅₋₇ heterocyclyl or together with R² an optionally substituted C₃₋₄ alkylene group wherein L³ and L⁴ are single bonds thus forming a C₅₋₆ ring fused with the aromatic ring to which L³ and L⁴ are attached;

R⁴ is H; or optionally substituted C₅₋₆ aryl or C₅₋₇ heterocyclyl;

L¹ is optionally substituted C₁₋₄ alkylene, C₅₋₆ arylene, C₁₋₄ alkylene-C₅₋₆ arylene or -L⁵N(R⁵)L⁶-, wherein L⁵ and L⁶ are independently selected from optionally substituted C₁₋₄ alkylene and C₅₋₆ arylene, and R⁵ is H or C₁₋₄ alkyl;

L² is a single bond; or optionally substituted C₁₋₄ alkylene or -L⁷C(═O)L⁸-, wherein L⁷ and L⁸ are independently selected from optionally substituted C₁₋₄ alkylene and a single bond; and

L³ and L⁴ are independently selected from a single bond, optionally substituted C₁₋₄ alkylene, -L⁹YN(OH)C(═O)L¹⁰- and -L⁹C(═O) N(OH)YL¹⁰-, wherein L⁹ and L¹⁰ are independently selected from optionally substituted C₁₋₄ alkylene, C₅₋₆ arylene, C₁₋₄ alkylene-C₅₋₆ arylene and a single bond, wherein Y is NH or a single bond;

or a pharmaceutically acceptable salt thereof for use in a method of therapy.

A second aspect of the present invention provides a pharmaceutical composition comprising a compound of formula I as defined in the first aspect or a pharmaceutically acceptable salt thereof together with a pharmaceutically acceptable carrier or diluent.

A further aspect of the present invention provides the use of a compound of formula I or a pharmaceutically acceptable salt thereof in the preparation of a medicament for the treatment of a condition alleviated by inhibition of glyoxalase I.

Another aspect of the present invention provides a method of treating a condition which can be alleviated by inhibition of glyoxalase I, which method comprises administering to a patient in need of treatment an effective amount of a compound of formula I, or a pharmaceutically acceptable salt thereof.

Another aspect of the present invention provides novel compounds or salts, solvates and chemically protected forms thereof, and methods of synthesis thereof as described herein.

In this aspect, the compounds are as provided in formula I wherein the compounds contain at least one —C(═O)N(OH)—group.

Conditions alleviated by inhibition of glyoxalase I are proliferative conditions. The term “proliferative condition” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g., histocytoma, glioma, astrocytoma, osteoma), cancers (e.g., lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposils sarcoma, melanoma), leukemias, psoriasis, bone diseases, fibroproliferative disorders (e.g., of connective tissues), and atherosclerosis.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

### DEFINITIONS

Cyano: The term “cyano”, as used herein, pertains to the monovalent moiety —CN.

Halo: The term “halo”, as used herein, pertains to the monovalent moiety —Y, wherein Y is a halogen atom. Examples of halo groups include —F, —Cl, —Br, and —I.

Hydroxy: The term “hydroxy”, as used herein, pertains to the monovalent moiety —OH.

Hydroxamic acid: The term “hydroxamic acid”, as used herein, pertains to the monovalent moiety —C(═O)NH(OH).

Sulfhydryl: The term “sulfhydryl”, as used herein, pertains to the monovalent moiety —SH.

C₁₋₄ alkyl group: The term “C₁₋₄ alkyl”, as used herein, pertains to a monovalent moiety obtained by removing a hydrogen atom from a carbon atom of a non-cyclic hydrocarbon compound having from 1 to 4 carbon atoms, and which may be saturated or unsaturated.

Examples of saturated C₁₋₄ alkyl groups include methyl (C₁); ethyl (C₂); propyl (C₃), which may be linear (n-propyl) or branched (iso-propyl); butyl (C₄), which may be linear (n-butyl) or branched (iso-butyl, sec-butyl and tert-butyl).

Examples of unsaturated C₁₋₄ alkyl groups, which may be referred to as C₁₋₄ alkenyl (if they included a double bond) or C₁₋₄ alkynyl (if they include a triple bond) groups, include ethenyl (vinyl, —CH═CH₂), ethynyl (ethinyl, —C≡CH), 1-propenyl (—CH═CH—CH₃), 2-propenyl (allyl, —CH—CH═CH₂), 2-propynyl (propargyl, —CH₂—C≡CH), isopropenyl (—C(CH₃)═CH₂) and butenyl (C₄).

C₃₋₇ Cycloalkyl: The term “C₃₋₇ cycloalkyl”, as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 ring atoms (unless otherwise specified).

Examples of saturated cycloalkyl groups include, but are not limited to, those derived from: cyclopropane (C₃), cyclobutane (C₄), cyclopentane (C₅), cyclohexane (C₆), cycloheptane (C₇), norbornane (C₇), norpinane (C₇), norcarane (C₇).

C₅₋₇ Heterocyclyl: The term “C₅₋₇ heterocyclyl”, as used herein, pertains to a monovalent moiety obtained by removing a hydrogen atom from a ring atom of a heterocyclic compound, which moiety has from 5 to 7 ring atoms, of which from 1 to 4 are ring heteroatoms.

In this context, the prefix C₅₋₇ denotes the number of ring atoms, or range of number of ring atoms, whether carbon atoms or heteroatoms. For example, the term “C₅₋₇ heterocyclyl” as used herein, pertains to a heterocyclyl group having 5 to 7 ring atoms. Examples of groups of heterocyclyl groups include C₅₋₇ heterocyclyl and C₅₋₆ heterocyclyl.

Examples of (non-aromatic) monocyclic heterocyclyl groups include, but are not limited to, those derived from:

N₁: pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇); O₁: oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and, N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of heterocyclyl groups which are also heteroaryl groups are described below with aryl groups.

C₅₋₆ aryl: The term “C₅₋₆ aryl”, as used herein, pertains to a monovalent moiety obtained by removing a hydrogen atom from an aromatic ring atom of an aromatic compound, which moiety has from 5 to 6 ring atoms (unless otherwise specified). In this context, the prefix C₅₋₆ denotes the number of ring atoms, or range of number of ring atoms, whether carbon atoms or heteroatoms. For example, the term “C₅₋₆ aryl,” as used herein, pertains to an aryl group having 5 or 6 ring atoms.

The ring atoms may be all carbon atoms, as in “carboaryl groups.” Examples of carboaryl groups include C₅₋₆ carboaryl, C₅ carboaryl, and C₆ carboaryl.

Examples of carboaryl groups include, but are not limited to, those derived from benzene (i.e., phenyl) (C₆).

Alternatively, the ring atoms may include one or more heteroatoms, as in “heteroaryl groups.” Examples of heteroaryl groups include C₅₋₆ heteroaryl, C₅ heteroaryl, and C₆ heteroaryl.

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₃S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Heterocyclic groups (including heteroaryl groups) which have a nitrogen ring atom in the form of an —NH— group may be N-substituted, that is, as —NR—. For example, pyrrole may be N-methyl substituted, to give N-methylpyrrole. Examples of N-substitutents include, but are not limited to C₁₋₄ alkyl, C₅₋₇ heterocyclyl, C₅₋₆ aryl, and acyl groups.

The term “bidentate substituents,” as used herein, pertains to substituents which have two points of covalent attachment, and which act as a linking group between two other moieties.

C₁₋₄ alkylene: The term “C₁₋₄ alkylene” as used herein, pertains to a bidentate moiety obtained by removing two hydrogen atoms from opposite ends of a linear hydrocarbon compound having from 1 to 4 carbon atoms (unless otherwise specified), and which may be saturated, partially unsaturated, or fully unsaturated. Thus, the term “alkylene” includes the sub-classes alkenylene, alkynylene, etc., discussed below.

In this context, the prefix C₁₋₄ denotes the number of carbon atoms, or range of number of carbon atoms. For example, the term “C₁₋₄ alkylene” as used herein, pertains to an alkylene group having from 1 to 4 carbon atoms.

Examples of saturated C₁₋₄ alkylene groups include, but are not limited to, —(CH₂)ₙ— where n is an integer from 1 to 4, for example, —CH₂— (methylene), —CH₂CH₂— (ethylene), —CH₂CH₂CH₂— (propylene), and —CH₂CH₂CH₂CH₂— (butylene)

Examples of partially unsaturated C₁₋₄ alkylene groups include, but are not limited to, —CH═CH— (vinylene), —CH═CH—CH₂—, —CH₂—CH═CH₂—, —CH═CH—CH₂—CH₂—, —CH═CH—CH═CH—.

C₅₋₆ arylene: The term “C₅₋₆ arylene”, as used herein, pertains to a bidentate moiety obtained by removing two hydrogen atoms, one from each of two different aromatic ring atoms of an aromatic compound, which moiety has from 5 to 6 ring atoms (unless otherwise specified).

In this context, the prefix C₅₋₆ denotes the number of ring atoms, or range of number of ring atoms, whether carbon atoms or heteroatoms. For example, the term “C₅₋₆ arylene” as used herein, pertains to an arylene group having 5 or 6 ring atoms. Examples of groups of arylene groups include C₅₋₆ arylene, C₅ arylene, and C₆ arylene.

The ring atoms may be all carbon atoms, as in “carboarylene groups” (e.g., C₅₋₆ carboarylene).

Examples of C₅₋₆ arylene groups which do not have ring heteroatoms (i.e., C₅₋₆ carboarylene groups) include, but are not limited to, those derived from the compounds discussed above in regard to carboaryl groups.

Alternatively, the ring atoms may include one or more heteroatoms, as in “heteroarylene groups” (e.g., C₅₋₆ heteroarylene).

Examples of C₅₋₆ heteroarylene groups include, but are not limited to, those derived from the compounds discussed above in regard to heteroaryl groups.

C₁₋₄ alkylene-C₅₋₆ arylene: The term “C₁₋₄ alkylene-C₅₋₆ arylene”, as used herein, pertains to a bidentate moiety comprising a C₁₋₄ alkylene moiety, —C₁₋₄ alkylene-, linked to a C₅₋₆ arylene moiety, —C₅₋₆ arylene-, that is, —C₁₋₄ alkylene-C₅₋₆ arylene-.

Examples of C₁₋₄ alkylene-C₅₋₆ arylene groups include, for example, methylene-phenylene, ethylene-phenylene, propylene-phenylene, and ethenylene-phenylene (also known as vinylene-phenylene).

The phrase “optionally substituted”, as used herein, pertains to a group, as above, which may be unsubstituted or which may be substituted by one of the following substituent groups or one of the groups listed above:

C₁₋₇ alkyl group: The term “C₁₋₇ alkyl”, as used herein, pertains to a monovalent moiety obtained by removing a hydrogen atom from a carbon atom of a hydrocarbon compound having from 1 to 7 carbon atoms (unless otherwise specified), which may be aliphatic or alicyclic, and which may be saturated, partially unsaturated, or fully unsaturated. Thus, the term “alkyl” includes the sub-classes alkenyl, alkynyl and cycloalkyl discussed below.

In this context, the prefixes (e.g. C₁₋₄, C₁₋₇, C₂₋₇, C₃₋₇, etc.) denote the number of carbon atoms, or range of number of carbon atoms. For example, the term “C₁₋₄ alkyl,” as used herein, pertains to an alkyl group having from 1 to 4 carbon atoms. Examples of groups of alkyl groups include C₁₋₄ alkyl (“lower alkyl”) and C₁₋₇ alkyl.

Examples of saturated alkyl groups include, but are not limited to, methyl (C₁), ethyl (C₂), propyl (C₃), butyl (C₄), pentyl (C₅), hexyl (C₆), heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

Cycloalkyl: The term “cycloalkyl”, as used herein, pertains to an alkyl group which is also a cyclyl group; that is; a monovalent moiety obtained by removing a hydrogen atom from an alicyclic ring atom of a cyclic hydrocarbon (carbocyclic) compound, which moiety has from 3 to 20 ring atoms (unless otherwise specified). Preferably, each ring has from 3 to 7 ring atoms.

Examples of saturated cycloalkyl groups include, but are not limited to, those derived from: cyclopropane (C₃), cyclobutane (C₄), cyclopentane (C₅), cyclohexane (C₆), cycloheptane (C₇), norbornane (C₇), norpinane (C₇), norcarane (C₇), adamantane (C₁₀), and decalin (decahydronaphthalene) (C₁₀).

Examples of saturated cycloalkyl groups, which are also referred to herein as “alkyl-cycloalkyl” groups, include, but are not limited to, methylcyclopropyl, dimethylcyclopropyl, methylcyclobutyl, dimethylcyclobutyl, methylcyclopentyl, dimethylcyclopentyl, methylcyclohexyl, and dimethylcyclohexyl, menthane, thujane, carane, pinane, bornane, norcarane, and camphene.

Examples of unsaturated cyclic alkenyl groups, which are also referred to herein as “alkyl-cycloalkenyl” groups, include, but are not limited to, methylcyclopropenyl, dimethylcyclopropenyl, methylcyclobutenyl, dimethylcyclobutenyl, methylcyclopentenyl, dimethylcyclopentenyl, methylcyclohexenyl, and dimethylcyclohexenyl.

Examples of cycloalkyl groups, with one or more other rings fused to the parent cycloalkyl group, include, but are not limited to, those derived from: indene (C₉), indan (e.g., 2,3-dihydro-1H-indene) (C₉), tetraline (1,2,3,4-tetrahydronaphthalene (C₁₀), acenaphthene (C₁₂), fluorene (C₁₃), phenalene (C₁₃), acephenanthrene (C₁₅), aceanthrene (C₁₆). For example, 2H-inden-2-yl is a C₅cycloalkyl group with a substituent (phenyl) fused thereto.

Alkenyl: The term “alkenyl,” as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of unsaturated alkenyl groups include, but are not limited to, ethenyl (vinyl, —CH═CH₂), 1-propenyl (—CH═CH—CH₃), 2-propenyl (allyl, —CH—CH′CH₂), isopropenyl (—C(CH₃)═CH₂), butenyl (C₄), pentenyl (C₅), and hexenyl (C₆).

Examples of unsaturated cyclic alkenyl groups, which are also referred to herein as “cycloalkenyl” groups, include, but are not limited to, cyclopropenyl (C₃), cyclobutenyl (C₄), cyclopentenyl (C₅), and cyclohexenyl (C₆)

Alkynyl: The term “alkynyl,” as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of unsaturated alkynyl groups include, but are not limited to, ethynyl (ethinyl, —C≡CH) and 2-propynyl (propargyl, —CH₂—C≡CH).

C₃₋₇ heterocyclyl group: The term “C₃₋₇ heterocyclyl”, as used herein, pertains to a monovalent moiety obtained by removing a hydrogen atom from a ring atom of a heterocyclic compound, which moiety has from 3 to 7 ring atoms (unless otherwise specified), of which from 1 to 4 are ring heteroatoms.

In this context, the prefixes (e.g. C₃₋₇, C₅₋₆ etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₇ heterocyclyl, C₅₋₇ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

C₅₋₇ aryl: The term “C₅₋₇ aryl” as used herein, pertains to a monovalent moiety obtained by removing a hydrogen atom from an aromatic ring atom of an aromatic compound, which moiety has from 5 to 7 ring atoms (unless otherwise specified).

In this context, the prefixes (e.g. C₅₋₇, C₅₋₆ etc.) denote the number of ring atoms, or range of number of ring atoms, whether carbon atoms or heteroatoms. For example, the term “C₅₋₆aryl” as used herein, pertains to an aryl group having 5 or 6 ring atoms. Examples of groups of aryl groups include C₅₋₇ aryl, C₅₋₆ aryl, C₅ aryl and C₆aryl.

The ring atoms may be all carbon atoms, as in “carboaryl groups”. Examples of carboaryl groups include C₅₋₇ carboaryl, C₅₋₆ carboaryl, C₅ carboaryl and C₆ carboaryl.

Examples of carboaryl groups include, but are not limited to, those derived from benzene (i.e. phenyl) (C₆).

Alternatively, the ring atoms may include one or more heteroatoms, as in “heteroaryl groups.” Examples of heteroaryl groups include C₅₋₇ heteroaryl, C₅₋₆ heteroaryl, C₅ heteroaryl and C₆ heteroaryl.

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Heterocyclic groups (including heteroaryl groups) which have a nitrogen ring atom in the form of an —NH— group may be N-substituted, that is, as —NR—. For example, pyrrole may be N-methyl substituted, to give N-methylpyrrole. Examples of N-substitutents include, but are not limited to C₁₋₇ alkyl, C₃₋₇ heterocyclyl, C₅₋₇ aryl, and acyl groups.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₇ heterocyclyl group (also referred to as a C₃₋₇ heterocyclyloxy group), or a C₅₋₇ aryl group (also referred to as a C₅₋₇ aryloxy group), preferably a C₁₋₇ alkyl group.

C₁₋₇ alkoxy: —OR, wherein R is a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Oxo (keto, -one): ═O.

Thione (thioketone): ═S.

Imino (imine): ═NR, wherein R is an imino substituent, for example, hydrogen, C₁₋₇ alkyl group, a C₃₋₇ heterocyclyl group, or a C₅₋₇ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₇ heterocyclyl group (also referred to as C₃₋₇ heterocyclylacyl), or a C₅₋₇ aryl group (also referred to as C₅₋₇ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═O)NH(OH).

Ester (carboxylate, carboxylic acid ester, oxycarbonyl): —C(═O)OR, wherein R is an ester substituent, for example, a C₁₋₇alkyl group, a C₃₋₇ heterocyclyl group, or a C₅₋₇ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇alkyl group, a C₃₋₇ heterocyclyl group, or a C₅₋₇aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₇ heterocyclyl group, or a C₅₋₇ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, hydrogen, a C₁₋₇ alkyl group, a C₃₋₇ heterocyclyl group, or a C₅₋₇ aryl group, preferably hydrogen or a C₁₋₇ alkyl group, and R² is an acyl substituent, for example, a C₁₋₇ alkyl group, a C₃₋₇ heterocyclyl group, or a C₅₋₇ aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

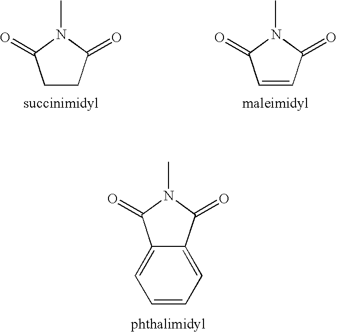

Thioamido (thiocarbamyl): —C(═S)NR¹R², wherein R¹ and R² are independently amino substituents, as defined for amino groups. Examples of amido groups include, but are not limited to, —C((═S)NH₂, —C((═S)NHCH₃, —C(═S)N(CH₃)₂, and —C(═S)NHCH₂CH₃.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₇ heterocyclyl group, or a C₅₋₇ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

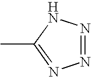

Amino: —NR¹R², wherein R³ and R² are independently amino substituents, for example, hydrogen, a C₁₋₇ alkyl group (also referred to as C₁₋₇ alkylamino or di-C₁₋₇ alkylamino), a C₃₋₇ heterocyclyl group, or a C₅₋₇ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amidine (amidino): —C(═NR)NR₂, wherein each R is an amidine substituent, for example, hydrogen, a C₁₋₇ alkyl group, a C₃₋₇ heterocyclyl group, or a C₅₋₇ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Cyano (nitrile, carbonitrile): —CN.

Isocyano: —NC.

Thiocyano (thiocyanato): —SCN.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₇ heterocyclyl group, or a C₅₋₇ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₇ heterocyclyl group, or a C₅₋₇ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₇ heterocyclyl group, or a C₅₋₇ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₇ heterocyclyl group, or a C₅₋₇ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph(phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₇ heterocyclyl group, or a C₅₋₇ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₇ heterocyclyl group, or a C₅₋₇ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₇ heterocyclyl group, or a C₅₋₇ aryl group, preferably a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₇ heterocyclyl group, or a C₅₋₇ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₇ heterocyclyl group, or a C₅₋₇ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH (CH₃), —S(═O)₂N(CH₃)₂, —S(═O)₂NH(CH₂CH₃, —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₇ heterocyclyl group, or a C₅₋₇ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇ alkyl group, a C₃₋₇ heterocyclyl group, or a C₅₋₇ aryl group, preferably a C₁₋₇ alkyl group. Examples of sulfinamino groups include, but are not limited to, —NHS(═O)CH₃ and —N(CH₃)S(═O)C₆H₅.

Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms such as esters. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Ester Derivatives

The carboxylic acid moiety of compounds of formula I may be protected as an ester for example, as an optionally substituted C₁₋₇ alkyl ester (e.g. a methyl ester; a t-butyl ester; a chloroethyl ester); an optionally substituted C₅₋₆ aryl ester (e.g. a phenyl ester; a chlorophenyl ester; a tolyl ester); or an optionally substituted C₁₋₄ alkylene-C₅₋₆ aryl ester (e.g., a benzyl ester; a nitrobenzyl ester). Thus included in the above are compounds of formula Ia: 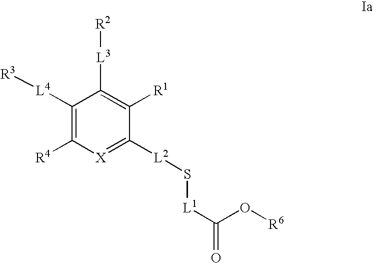
wherein R¹, R², R³, R⁴, L¹, L², L³and L⁴ are as defined above and R⁶ is selected from optionally substituted C₁₋₇ alkyl, C₅₋₆ aryl and C₁₋₄ alkylene-C₅₋₆ aryl.

C₁₋₄ alkylene-C₅₋₆ aryl: The term “C₁₋₄ alkylene-C₅₋₆ aryl”, as used herein, pertains to a bidentate moiety comprising a C₁₋₄ alkylene moiety, —C₁₋₄ alkylene-, linked to a C₅₋₆ aryl moiety, —C₅₋₆ aryl, that is, —C₁₋₄ alkylene-C₅₋₆ aryl.

Examples of C₁₋₄ alkylene-C₅₋₆ aryl groups include, for example, methylene-phenyl (also known as benzyl), ethylene-phenyl, propylene-phenyl, and ethenylene-phenyl (also known as vinylene-phenylene).

The ester derivatives of formula Ia may function as prodrugs for the treatment of conditions alleviated by inhibition of glyoxalase I, i.e. proliferative conditions.

Isomers, Salts, Solvates and Protected Forms

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

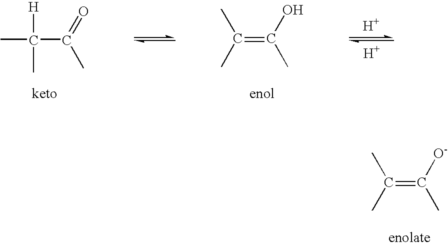

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

A wide variety of such “protecting”, “blocking”, or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH—Cbz); as a t-butoxy amide (—NHCO—OC(CH₃)₃, —NH—Boc); a 2-biphenyl-2-propoxy amide (—NHCO—OC(CH₃)₂C₆H₄C₆H₅, —NH—Bpoc), as a 9-fluorenylmethoxy amide (—NH—Fmoc), as a 6-nitroveratryloxy amide (—NH—Nvoc), as a 2-trimethylsilylethyloxy amide (—NH—Teoc), as a 2,2,2-trichloroethyloxy amide (—NH—Troc), as an allyloxy amide (—NH-Alloc), as a 2(-phenylsulfonyl)ethyloxy amide (—NH—Psec); or, in suitable cases (e.g., cyclic amines), as a nitroxide radical (>N—O—).

For example, a carboxylic acid group may be protected as an ester for example, as: an C₁₋₇ alkyl ester (e.g., a methyl ester; a t-butyl ester); a C₁₋₇haloalkyl ester (e.g., a C₁₋₇trihaloalkyl ester); a triC₁₋₇ alkylsilyl-C₁₋₇ alkyl ester; or a C₅₋₇ aryl-C₁₋₇ alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. Suitable dose ranges will typically be in the range of from 0.01 to 20 mg/kg/day, preferably from 0.1 to 10 mg/kg/day.

Compositions and their Administration

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

Acronyms

For convenience, many chemical moieties are represented using well known abbreviations, including but not limited to, methyl (Me), ethyl (Et), n-propyl (nPr), iso-propyl (iPr), n-butyl (nBu), sec-butyl (sBu), iso-butyl (iBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), acetonitrile (ACN), trifluoroacetic acid (TFA), dimethylformamide (DMF), tetrahydrofuran (THF), and dimethylsulfoxide (DMSO).

General Synthesis Methods

Methods for the chemical synthesis of compounds of the present invention are described herein. These methods may be modified and/or adapted in known ways in order to facilitate the synthesis of additional compounds within the scope of the present invention. Descriptions of general laboratory methods and procedures, useful for the preparation of the compounds of the present invention, are described in Vogel's Textbook of Practical Organic Chemistry (5^{th} edition, Ed. Furniss, B. S., Hannaford, A. J., Smith, P. W. G., Tatchell, A. R., Longmann, UK).

In the methods described below, other substituent groups to those introduced may be present as precursors of those groups, or as protected versions of those groups.

Compounds of formula I where L² is —C(═O)—CH₂—, can be synthesised according to the route shown in Scheme 1.

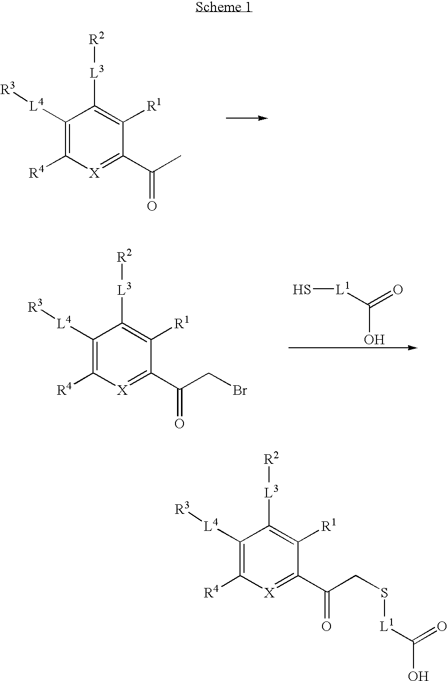

Compounds of formula I where X is N, L₂ is a single bond, R₁═CN and R₃═H can be synthesised according to the route based on those disclosed in Manna et al., Bioorg. Med. Chem. Lett. 10, 1883-1885 (2000) and Salman, Pharmazie 54, 178-183 (1999) (scheme 2).

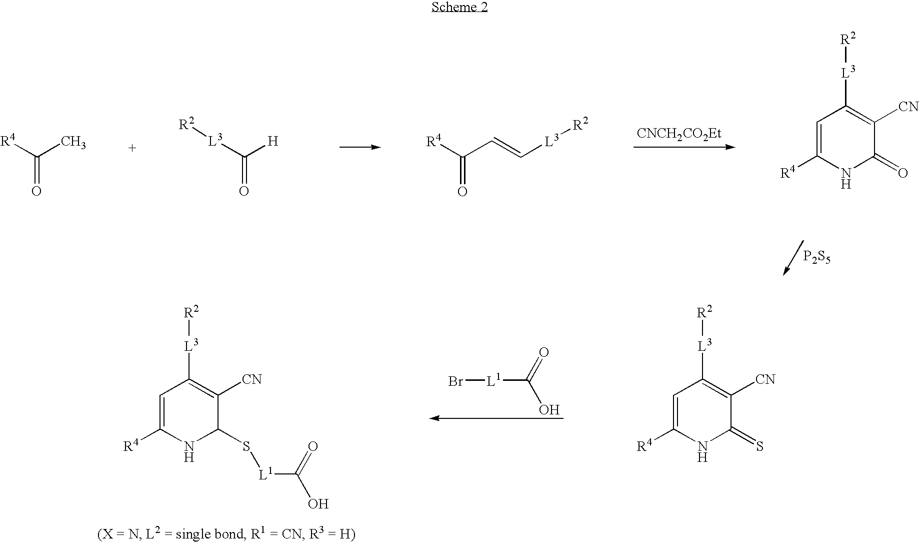

Compounds of formula I where L₂ is a single bond and L₄=—CH₂N(OH)C(═O)— can be synthesised according to the route based on that shown in Scheme 3. Compounds of formula I where L₂ is a single bond and L₃=—CH₂N(OH)C(═O)— can also be synthesised by a route based on that shown in Scheme 3, except that the starting material comprises a hydroxymethyl group present in the meta position relative to the thiol group, rather than in the para position as shown in scheme 3.

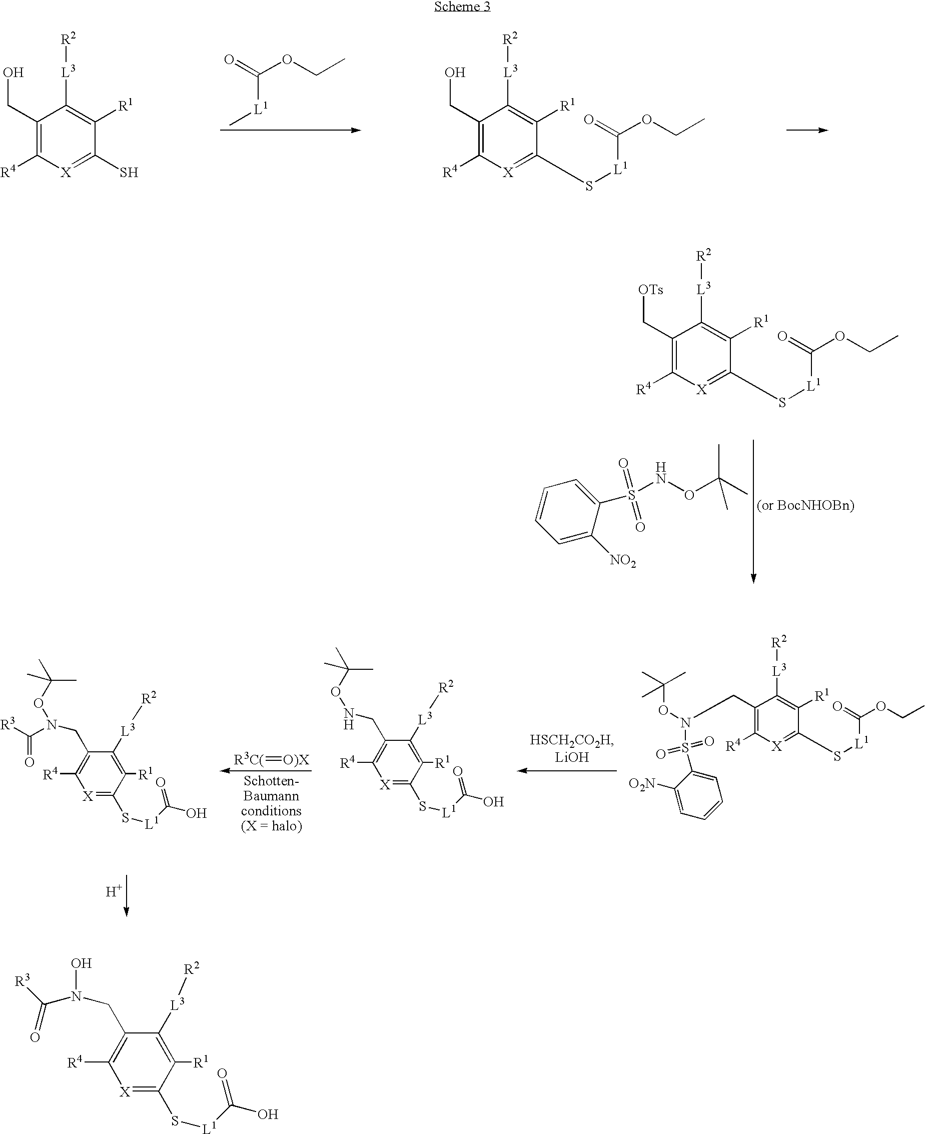

As shown in the above three schemes, the sulfur atom between L² and L¹ may be introduced as a nucleophilic attacking group (scheme 1) or by replacement (scheme 2), or may be present in the starting material (scheme 3).

As an alternative to the substitution of the doubly protected amine group at the position meta to the X group in scheme 3, a singly protected amine group may be substituted in the same position. In this case the group meta to the X group on the heterocyclic compound may be an —OTs group (as in scheme 3) or may alternatively be an —OH group. This general reaction is shown in scheme 4 below. In either case, the reaction may proceed by reaction with a singly protected amine group. Subsequent substitution of the amine —H group with R³C(═O)X may then be achieved as shown in scheme 3 followed by deprotection of the amine groups to leave —OH attached to the amine N atom.

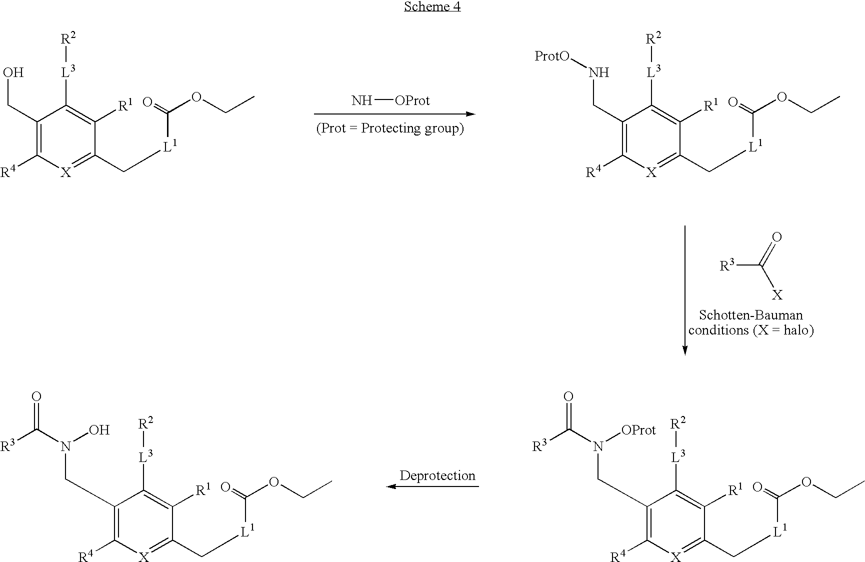

In scheme 4 above, the protecting group may be any suitable protecting group such as acetyl, allyl, alloc, BOM, benzyl, benzoyl, DMPM, FMOC, MEM, MOM, MPM, PMB, PMP, SEM, TBDMS, TBDPS, TBS, THP, TIPS, TMS, trityl or tosyl.

In general, the group R³ can be derived using standard reactions for the conversion of aryl substituent groups, including alkylation, reduction and substitution.

If R³ and R⁴ form a fused ring, then this would be present in the starting materials of a synthesis route to the compounds of the present invention.

When R⁴ is an aryl or heterocyclyl group, this may be introduced to the compound by means of Suzuki coupling, i.e. by the coupling of an aryl halide to an organoboron derivative (scheme 5, wherein L² indicates -L²-S-L¹-CO₂H or a precursor or protected form thereof and R is aryl or alkyl):

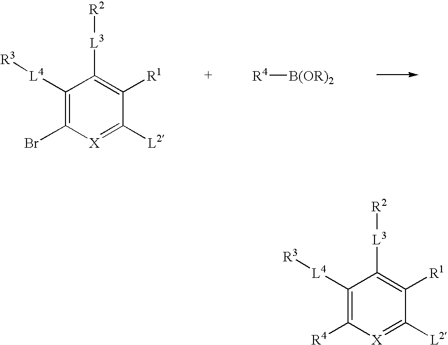

A similar approach may be used to couple R³ and R² to the central ring, when L⁴ and L³ respectively are single bonds. Furthermore, if R² or R³ are aryl groups, the appropriate aryl halides may be coupled to boron derivatives of the remainder of the compound.

Certain compounds of the present invention are commercially available or can be derived from such compounds.

Preferences

The following preferences may be combined with one another, and may be different for each aspect of the present invention.

R¹ is preferably H, cyano, methyl, halo, hydroxy, hydroxamic acid, methoxy, amino, methylamino, dimethylamino, nitro, sulfhydryl, or methyl sulfide.

More preferably R¹ is cyano, H or hydroxamic acid.

Preferably L¹ is phenylene, methylene, ethylene, —CH(CH₃)—, —CH(ⁱPr)—, —CH(Ph)—, —CH₂-phenylene-, —CH₂C(═O)NHCH₂— or —CH₂C(═O)NH-phenylene-.

Preferably L² is a single bond or —C(═O)CH₂—.

Preferably L³ is a single bond, -L⁹YN(OH)C(═O)L¹⁰- or -L⁹C(═O)N(OH)YL¹⁰-, wherein L⁹ and L¹⁰ are independently selected from optionally substituted C₁₋₄ alkylene, C₅₋₆ arylene, C₁₋₄ alkylene-C₅₋₆ arylene and a single bond, and wherein Y is NH or a single bond.

Preferably L⁴ is a single bond, -L⁹YN(OH)C(═O)L¹⁰- or -L⁹C(═O)N(OH)YL¹⁰-, wherein L⁹ and L¹⁰ are independently selected from optionally substituted C₁₋₄ alkylene, C₅₋₆ arylene, C₁₋₄ alkylene-C₅₋₆ arylene and a single bond, and wherein Y is NH or a single bond.

For example, L³ or L⁴ may be a single bond, —CH₂N(OH)C(═O)—, -phenylene-CH₂N(OH)C(═O)—, -phenylene-NHN(OH)C(═O)—, or —CH₂C(═O)N(OH)—.

When X is CH, preferably one or more of R¹, R² and R⁴ are H. More preferably two of R¹, R² and R⁴ are H, when X is CH. It is most preferred that all of R¹, R² and R⁴ are H, when X is CH.

When X is CH, preferably one of R² and R³ is optionally substituted C₅₋₆ aryl, C₃₋₇ cycloalkyl or C₅₋₇ heterocyclyl. More preferably, when X is CH, R³ is optionally substituted C₅₋₆ aryl, C₃₋₇ cycloalkyl or C₅₋₇ heterocyclyl. It is most preferred that when X is CH, R³ is optionally substituted phenyl or C₃₋₇ cycloalkyl. For example, R³ may be phenyl or cyclopentyl.

When X is CH, preferably L¹ is phenylene or —CH(Ph)—.

When X is CH, preferably one of L³ and L⁴ is a single bond. More preferably, when X is CH, L³ is a single bond.

When X is CH, preferably one of L³ and L⁴ is -L⁹YN(OH)C(═O)L¹⁰- or -L⁹C(═O)N(OH)YL¹⁰-, wherein L⁹ and L¹⁰ are independently selected from optionally substituted C₁₋₄ alkylene, C₅₋₆ arylene, C₁₋₄ alkylene-C₅₋₆ arylene and a single bond, and wherein Y is NH or a single bond. More preferably, when X is CH, L⁴ is -L⁹YN(OH)C(═O)L¹⁰- or -L⁹C(═O)N(OH)YL¹⁰-, wherein L⁹ and L¹⁰ are independently selected from optionally substituted C₁₋₄ alkylene, C₅₋₆ arylene, C₁₋₄ alkylene-C₅₋₆ arylene and a single bond, and wherein Y is NH or a single bond.

When X is N, R¹ is preferably CN or hydroxamic acid.

When X is N, R² is preferably selected from optionally substituted C₅₋₆ aryl, C₅₋₇ heterocyclyl, CF₃ and, together with R³, an optionally substituted butylene group wherein L³ and L⁴ are single bonds thus forming a C₆ ring fused with the aromatic ring to which L³ and L⁴ are attached.

When X is N, R² is more preferably selected from optionally substituted C₅₋₆ aryl and C₅₋₇ heterocyclyl.

When X is N, R² is even more preferably optionally substituted phenyl or thiophenyl. For example, when X is N, R² may be thiophenyl, phenyl, p-chlorophenyl, p-methoxyphenyl, o-methoxyphenyl, p-fluorophenyl. When X is N and R² is a monosubstituted phenyl, it is preferred that R² is a parasubstituted phenyl.

When X is N preferably R³ is H or, together with R², an optionally substituted butylene group wherein L³ and L⁴ are single bonds thus forming a C₆ ring fused with the aromatic ring to which L³ and L⁴ are attached.

When X is N, it is more preferable that R³ is H and L⁴ is a single bond such that the compounds of the invention are of formula Ib.

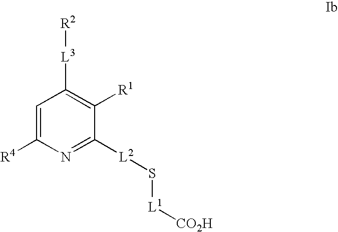

When X is N, R⁴ is preferably selected from optionally substituted C₅₋₆ aryl and C₅₋₇ heterocyclyl. When X is N, R⁴ is more preferably optionally substituted phenyl, thiophenyl, furanyl or pyridyl. For example, when X is N R⁴ may be phenyl, p-tolyl, p-chlorophenyl, p-methoxyphenyl, 3,4-dimethoxyphenyl, p-fluorophenyl, thiophenyl, furanyl or pyridyl. When X is N and R⁴ is a monosubstituted phenyl, it is preferable that R⁴ is a parasubstituted phenyl. When X is N and R⁴ is a disubstituted phenyl, it is preferred that the substituents are in the meta and para positions.

When R², R³ or R⁴ is a substituted C₅₋₆ aryl group, preferred substituents are halo, C₁₋₄ alkyl or —OR, wherein R is C₁₋₄ alkyl. When R², R³ or R⁴ is a monosubstituted phenyl group it is preferred that the substituent is in the para position. When R², R³ or R⁴ is a disubstituted phenyl group it is preferred that the substituents are in the para and meta positions. For example R², R³ and R⁴ may be p-tolyl, p-chlorophenyl, p-methoxyphenyl, 3,4-dimethoxyphenyl, p-fluorophenyl.

When compounds of formula I have at least one —C(═O)N(OH)-group, preferably at least one of R¹, L³ or L⁴ comprises a —C(═O)N(OH)— group. Preferably L⁴ comprises a —C(═O)N(OH)-group.

When compounds of formula I have at least one —C(═O)N(OH)-group, it is preferable that L⁴ is a L⁹-C(═O)N(OH)-group where preferably L⁹ is selected from C₁₋₄ alkylene and C₅₋₆ arylene and most preferably L⁹ is phenyl.

When compounds of formula I have at least one —C(═O)N(OH)-group, it is preferable that X is CH.

When compounds of formula I have at least one —C(═O)N(OH)-group, preferably at least one, more preferably at least two of R¹, R² and R⁴ is H. Most preferably all of R¹, R² and R⁴ are H.

When compounds of formula I have at least one —C(═O)N(OH)-group, R³ is preferably C₅₋₆ aryl and more preferably R³ is phenyl.

When compounds of formula I have at least one —C(═O)N(OH)-group, R⁶ is preferably H or C₁₋₇ alkyl and is more preferably C₁₋₃ alkyl.

When compounds of formula I have at least one —C(═O)N(OH)-group, L¹ is preferably phenylene, —CH(Ph)—, —CH₂-phenylene- or —CH₂C(═O)NH-phenylene-.

When compounds of formula I have at least one —C(═O)N(OH)-group, L² is preferably a single bond or —C(═O)CH₂—.

When compounds of formula I have at least one —C(═O)N(OH)-group, L³ is preferably a single bond.

Particularly preferred compounds include those listed in tables 1 and 4.

TABLE 1

| Com- | |
|---|---|
| pound | Structure |
| A | 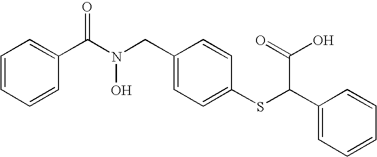 |
| B | 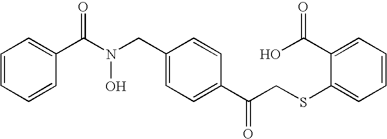 |
| C | 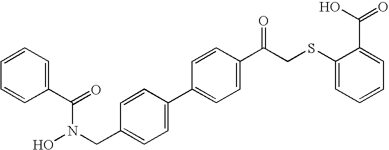 |
| D | 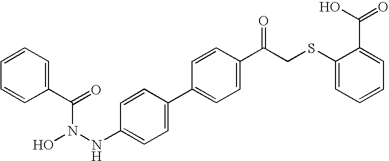 |
| E | 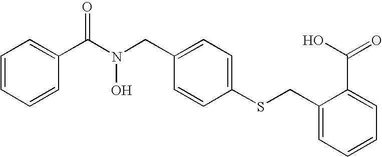 |
| F | 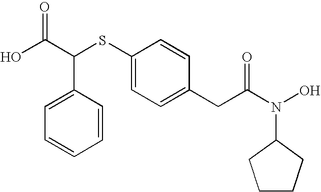 |
| G | 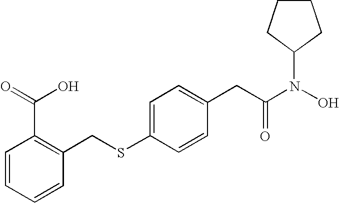 |

### EXAMPLES

### Example 1

### Formation of {4-[(Benzoyl-hydroxy-amino)-methyl]-phenylsulfanyl}-phenyl-acetic acid ethyl ester (iv)

### Step 1—(4-Hydroxymethyl-phenylsulfanyl)-phenyl-acetic acid ethyl ester (i)

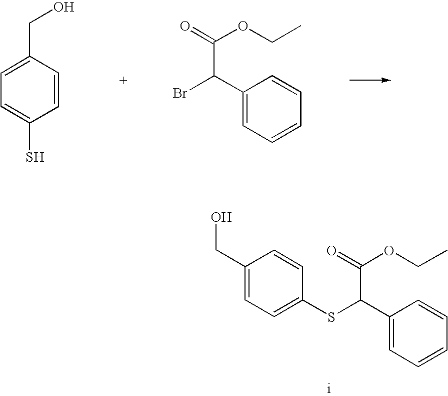

4-Mercaptobenzyl alcohol (0.582 g, 0.0042 mol), ethyl alpha bromophenyl acetate (0.727 ml, 0.0042 mol) and potassium carbonate (0.86 g, 0.0062 mol, 1.5 eq) were refluxed in acetone (25 ml) for 12 h. The crude material was purified by flash column chromatography (Ethyl acetate/hexane) to give the product i as a yellow oil (0.79 g, 63%).

### Step 2—(4-Methylaminomethyl-phenylsulfanyl)-phenyl-acetic acid ethyl ester (ii)

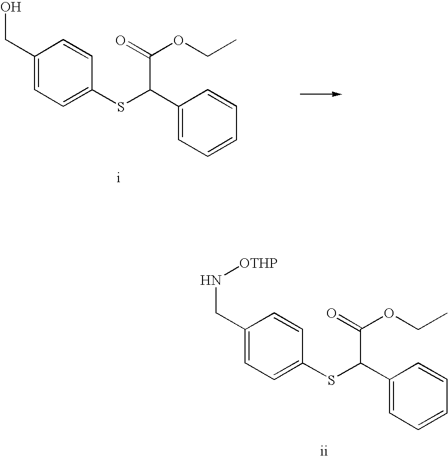

Trifluoroacetic anhydride (0.4 ml, 0.002 mol) was added to a solution of i (0.65 g, 0.002 mol) in dichloromethane at 0° C. under nitrogen. After 5 min lutidine (0.29 ml, 0.0024 mol) was added and the solution stirred for a further 5 min. O-Tetrahydro-2H-pyran-2-yl-hydroxylamine (0.5 g, 0.004 mol, 2 eq) was added and the cooling removed. The reaction was stirred at room temperature overnight. The required product was isolated following flash column chromatography yielding ii (362 mg, 42%), m/z [ES] 402 [M+H]⁺ 424 [M+Na]⁺

### Step 3—{4-[(Benzoyl-benzoyloxy-amino)-methyl]-phenylsulfanyl}-phenyl-acetic acid ethyl ester (iii)

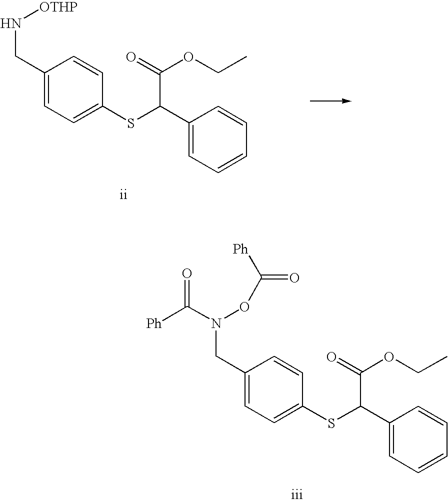

To a solution of ii (362 mg, 0.9 mmol) and triethylamine (0.19 ml, 1.5 eq) in dichloromethane (30 ml) was added benzoyl chloride (0.16 ml, 1.5 eq). This was allowed to stir at room temperature for 2 h. The solvent was removed in situ and the product purified by flash column chromatography (EtOAc/hexane). The unexpected compound iii was recovered (0.147 g, 31%) as a colourless oil, m/z [ES] 548 [M+Na]⁺

### Step 4—(4-[(Benzoyl-hydroxy-amino)-methyl]-phenylsulfanyl)-phenyl-acetic acid ethyl ester (iv)

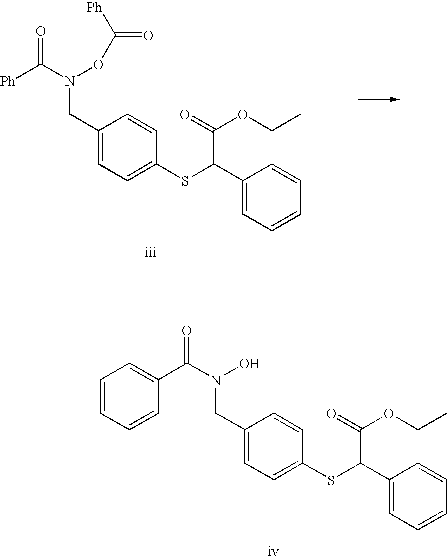

To a solution of iii (0.144 g, 0.27 mmol) in dichloromethane (10 ml) was added polymer supported trisamine (2.46 mmol/g, 0.33 g, 0.82 mmol, 3 eq). The reaction was stirred at room temperature for 72 h. The resin was filtered off and the residue concentrated in vacuo. The crude material was purified by prep HPLC to yield the required product (iv) (47 mg, 41%). 1H NMR (400 MHz, MeOD-d4) δ: 7.7-7.1 (14H, Ar), 4.95 (1H, s), 4.75 (2H, m, CH₂), 3.95 (2H, m), 0.95 (3H, t), m/z [ES] 422 [M+H]⁺

### Example 2

### Formation of {4-[(Benzoyl-hydroxy-amino)-methyl]-phenylsulfanyl)-phenyl-acetic acid (A)

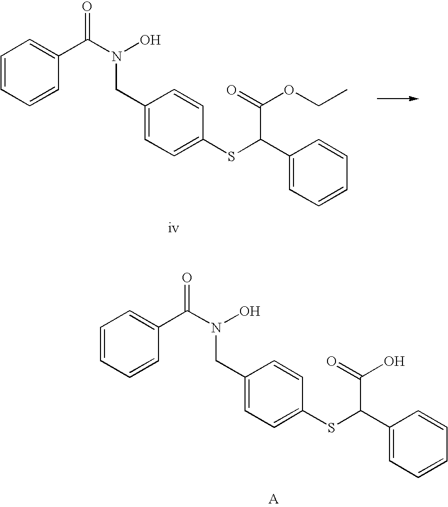

To a solution of iv (0.079 g, 0.19 mmol) in THF/water (6 ml/2 ml) was added sodium hydroxide (0.47 mmol, 2.5 eq). The reaction was stirred at room temperature for 16 h. The solution was neutralized with 1M HCl (0.11 ml) and the solvent removed in vacuo. The crude material was purified by prep HPLC to yield the required product (A) (4.1 mg, 6%).

1H NMR (400 MHz, MeOD-d4) δ: 7.7-7.1 (14H, Ar), 4.9 (1H, s), 4.75 (2H, m, CH₂), m/z [ES] 394 [M+H]⁺

### Example 3

### Formation of 2-{4-[(Benzoyl-hydroxy-amino)-methyl]-phenylsulfanylmethyl}-benzoic acid methyl ester

### Step 1—2-Bromomethyl-benzoic acid methyl ester

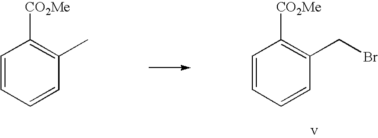

To a solution of methyl 2-methylbenzoate (5 g, 0.033 mol) in carbon tetrachloride (85 ml) was added n-bromosuccinimide (5.93 g, 0.033 mol) and benzoyl peroxide (0.22 g, 0.9 mol). The reaction was refluxed for 4 hr. The reaction was cooled to room temperature. The white precipitate was filtered and the solvent removed. The oil was dissolved in Et₂O and cooled to −78° C. The product precipitated and collected yielding v (5.86 g, 77%).

### Step 2—2-(4-Hydroxymethyl-phenylsulfanylmethyl)-benzoic acid methyl ester

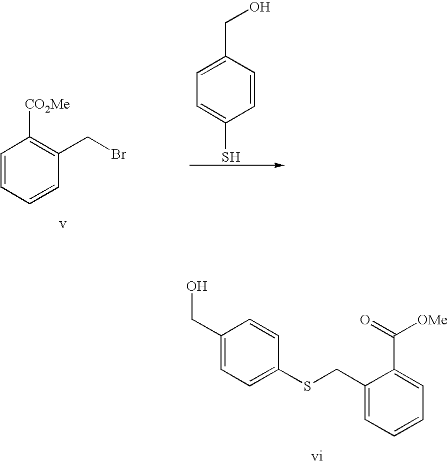

4-Mercaptobenzyl alcohol (0.579 g, 0.0041 mol), methyl 2-bromomethyl benzoate (v) (0.946, 0.0041 mol) and potassium carbonate (0.85 g, 0.0062 mol, 1.5 eq) were refluxed in acetone (25 ml) for 12 h. The crude material was purified by flash column chromatography (ethyl acetate/hexane) to give the product vi as a colourless oil (0.855 g, 72%), m/z [ES] 311 [M+Na]⁺

### Step 3—2-(4-[(Tetrahydro-pyran-2-yloxyamino)-methyl]-phenylsulfanylmethyl}-benzoic acid methyl ester

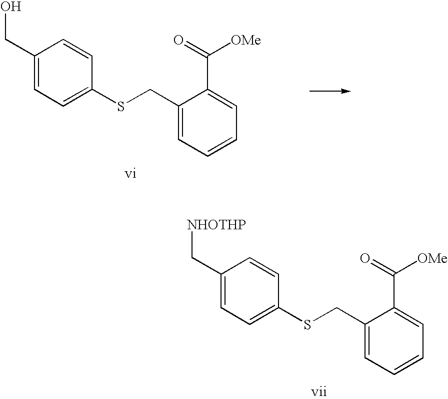

Trifluoroacetic anhydride (0.91 ml, 0.005 mol) was added to a solution of vi (1.41 g, 0.005 mol) in dichloromethane at 0° C. under nitrogen. After 5 min lutidine (0.66 ml, 0.56 mol) was added and the solution stirred for a further 5 min. O-Tetrahydro-2H-pyran-2-yl-hydroxylamine (1.15 g, 0.0098 mol, 2 eq) was added dropwise and the reaction stirred for 30 minutes. The required product was isolated following prep HPLC yielding vii (0.114 g, 6%), m/z [ES] 388 [M+H]⁺ 410 [M+Na]⁺

### Step 4—2-(4-{[Benzoyl-(tetrahydro-pyran-2-yloxy)-amino]-methyl}-phenylsulfanylmethyl)-benzoic acid methyl ester

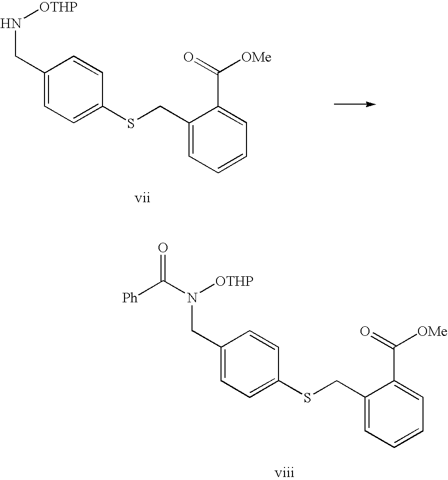

To a solution of vii (114 mg, 0.29 mmol) and diisopropylethylamine (0.19 ml, 1.5 eq) in dichloromethane (30 ml) was added benzoyl chloride (0.04 ml, 1.2 eq). This was allowed to stir at room temperature for 1 h. The reaction was quenched with aqueous NaHCO₃ (1 ml), dried (Na₂SO₄) and the solvent removed in vacuo. The product was purified by flash column chromatography (EtOAc/hexane). The required product viii was recovered (96.5 mg, 67%) as a colourless oil, m/z [ES] 492 [M+H]⁺, 514 [M+Na]⁺

### Step 5—2-{4-[(Benzoyl-hydroxy-amino)-methyl]-phenylsulfanylmethyl}-benzoic acid methyl ester

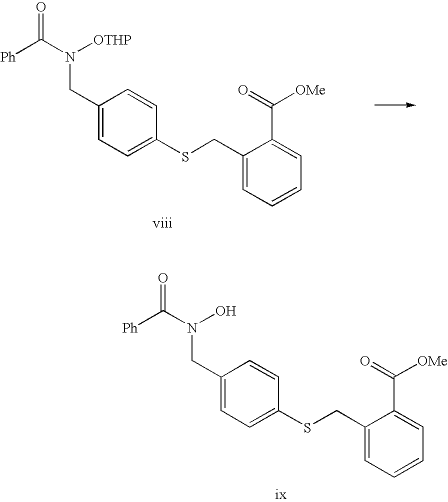

Compound viii (68.2 mg, 0.14 mmol) was stirred with (TFA/H₂O/DCM, 2.5:1:96.5, 10 ml) at room temperature and monitored by LC-MS (reaction was complete ˜3 h). The reaction mixture was quenched with aqueous NaHCO₃. The phases were separated and the organic layer concentrated in vacuo after drying. Compound ix required no further purification (97% LC-MS). 1H NMR (400 MHz, MeOD-d4) δ: 7.75 (1H, d, Ar), 7.5-7.6 (2H, m, Ar), 7.45-7.1 (10H, m, Ar), 4.75 (2H, m), 4.4 (2H, s), 3.75 (3H, s), m/z [ES) 408 (M+H]⁺.

### Example 4

### Formation of 2-{4-[(Benzoyl-hydroxy-amino)-methyl]-phenylsulfanylmethyl}-benzoic acid (E)

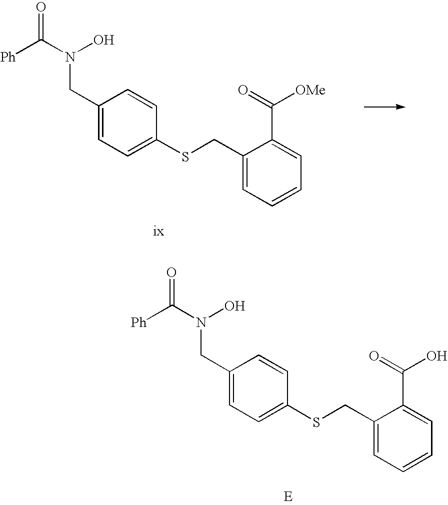

To a solution of ix (60 mg, 0.15 mmol) in MeOH/water (4 ml/2 ml) was added NaOH (0.22 ml, 1M solution, 0.22 mmol) and the reaction stirred at room temperature for 6 days. After this time 60% conversion to product was observed. The required product was isolated following prep HPLC yielding the required product (E) as a white solid (7 mg, 12%). 1H NMR (400 MHz, MeOD-d4) δ: 7.85 (1H, m, Ar), 7.5-7.6 (2H, m, Ar), 7.45-7.1 (10H, m, Ar), 4.75 (2H, m), 4.45 (2H, s), m/z [ES] 394 [M+H]⁺.

### Example 5

### Glyoxalase I Inhibition Assay

Glyoxalase I catalyses the formation of S-D-lactoylglutathione from the hemithioacetal that forms non-enzymatically from methylglyoxal and reduced glutathione (GSH). The standard literature assay is a cuvette-based spectrophotometric method using the product of glyoxalase I, S-D-lactoylglutathione, as the chromophore (240 nm) (Racker, J. Biol. Chem. 190, 685-686 (1951); Principato et al., Biochem International 6, 249-255 (1983)). This literature method was modified for use as a 96-well plate, kinetic assay.

The buffer (0.1M potassium phosphate buffer pH 6.6) and inhibitors are added to appropriate wells of a 96 well plate. Methylglyoxal (prepared in potassium phosphate buffer pH 6.6) and reduced glutathione (prepared in potassium phosphate buffer pH 6.6) are added to appropriate wells. These reagents are incubated for 15 minutes, shaking at room temperature to allow the formation of the hemithioacetal substrate of glyoxalase I. Recombinant human glyoxalase I (expressed in E. coli. and purified by S-hexylglutathione-agarose chromatography as described in Ridderstorm M. and Mannervik B., Biochem J. 314, 463-467 (1996)) is added and the plate is shaken briefly, before being placed in a Spectra Max 190 microplate spectrophotometer (Molecular Devices) at 25° C. Absorbance is monitored at 240 nm, with readings being taken every 30 seconds. The reaction is monitored for 15 minutes and PathCheck® measurements are taken on completion of the assay and the absorbance values normalized to a 1 cm pathlength. The integral software determines the Vmax using the first 20 readings. S-hexylglutathione is used as a positive control in the assays.

Data are expressed as a percentage of the control Vmax, measured in the absence of inhibitor.

Compounds iv, A, ix and E from examples 1 to 4 above respectively were subjected to the above glyoxalase I binding assay at a concentration of 20 μM (20 micromolar). Inhibitory effects are expressed in table 2 as either IC₅₀ values, defined as the concentration, in μM, of the compound that results in 50% of the vehicle control response, or as the percentage of the vehicle control response at the highest concentration of compound tested.

TABLE 2

| Compound | % inhibition at 20 μM | IC₅₀ (μM) |
|---|---|---|
| iv | 28.5 | |
| A | | 9.7 |
| ix | 0 | |
| E | | 1.9-2.26 |

The results in table 2 show that compunds A and E exhibit good glyoxalase I inhibition, i.e. have low IC₅₀ values, whereas the corresponding ethyl and methyl esters, iv and ix respectively, show relatively low % inhibition of glyoxalase I activity. This indicates that the ester forms of these compounds do not inhibit glyoxalase I as well as the free forms so indicating the suitability of the ester forms as a prodrug.

### Example 6

### HL60 Cell Assay

HL60 (human promyelocytic leukaemia) cells were seeded (50 μl/well) at a density between 0.25-0.4×10⁵/ml in a 96 multi-well plate. Twenty four hours later 50 μl. of the relevant compound dilution made up in culture medium was added to the wells (final concentration range 20-1.25 μM in 0.1% DMSO). After incubating at 37° C. in a 5% CO₂ atmosphere for a further 72 hours, assessment of growth inhibitory effects of compounds was measured using a colorimetric assay that is based on the cleavage of the tetrazolium salt WST-1 (Roche) by mitochondrial dehydrogenase in viable cells. WST-1 reagent (10 μl) was added to each well and the plate was agitated for 1 min. After a 3-4 hour incubation at 37° C. in a 5% CO₂ incubator the absorbance at 450 nM was read spectrophotometrically, after subtraction of a reference wavelength absorbance, at 690 nM.

Compounds iv, ix and E from examples 1, 3 and 4 above respectively were subjected to the above cell assay. Results were the mean of six replicates and were expressed as the percentage of the DMSO vehicle control response. Inhibitory effects are expressed in table 3 in the form of either IC₅₀ values, which was defined as the concentration (μM) of compound that results in 50% of the vehicle control response, or as the % of vehicle control response at the highest concentration of compound tested.

TABLE 3

| | % proliferation | |
|---|---|---|
| Compound | inhibition in HL60s | IC₅₀ (μM) |
| iv | 80 | 8.3 |
| ix | | 15 |
| E | 4 | |

Without wishing to be bound by theory, the significant activity of the ester compounds iv and ix in the HL60 assay, shown in table 3, indicates that the ester compounds may be converted into active form in cell culture hence demonstrating their suitability for use as prodrug compounds.

### Example 7

### Glyoxalase Binding Assay of Commercially Available Compounds

The compounds of table 4 were obtained from commercial sources.

TABLE 4

| Com- | |
|---|---|
| pound | Structure |
| 1 | 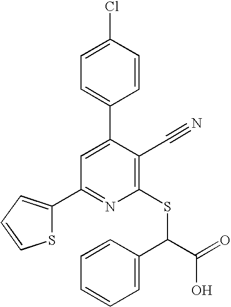 |
| 2 | 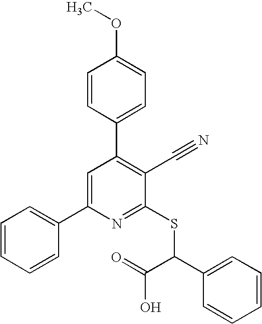 |
| 3 | 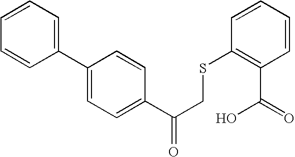 |
| 4 | 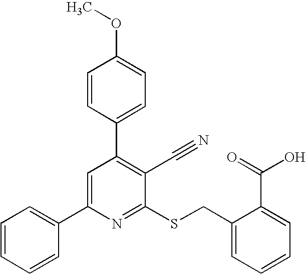 |
| 5 | 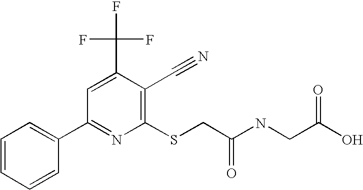 |
| 6 | 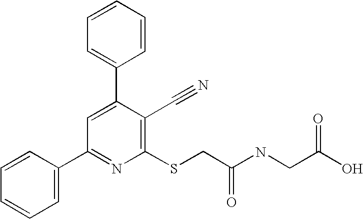 |
| 7 | 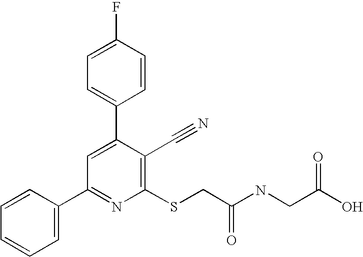 |
| 8 | 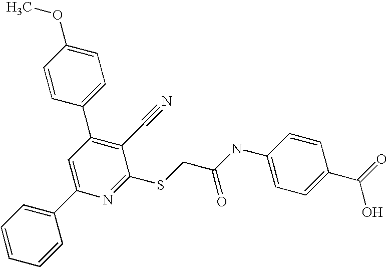 |
| 9 | 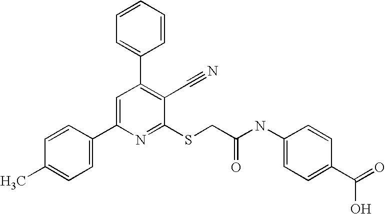 |
| 10 | 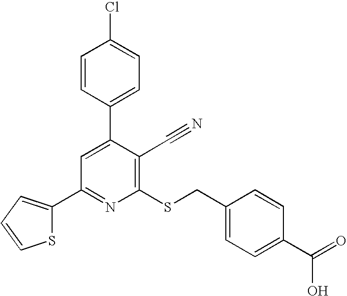 |
| 11 | 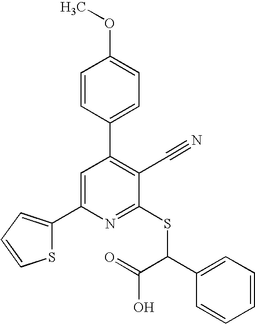 |
| 12 | 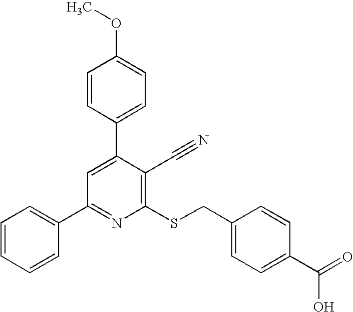 |
| 13 | 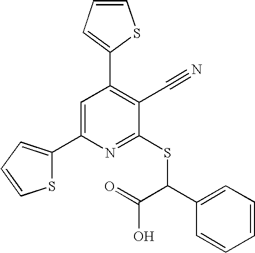 |
| 14 | 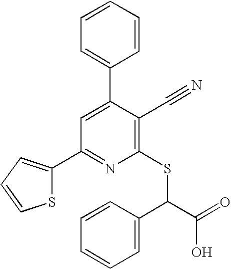 |
| 15 | 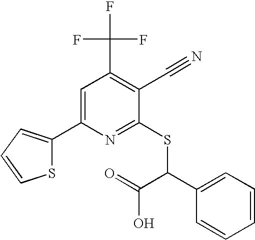 |
| 16 | 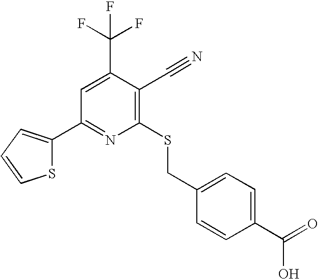 |
| 17 | 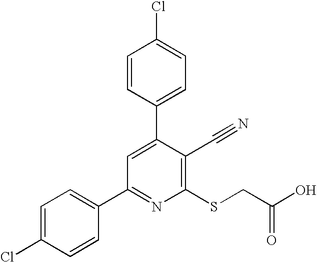 |
| 18 | 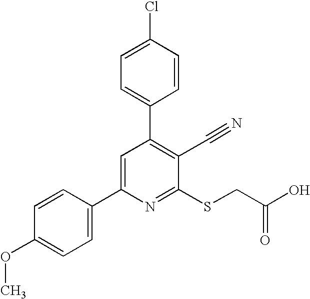 |
| 19 | 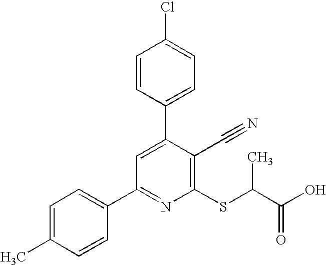 |
| 20 | 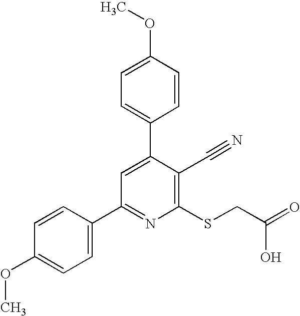 |
| 21 | 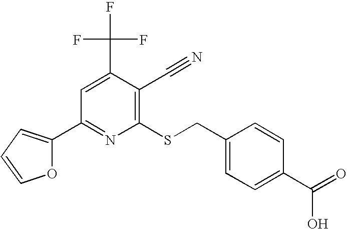 |
| 22 | 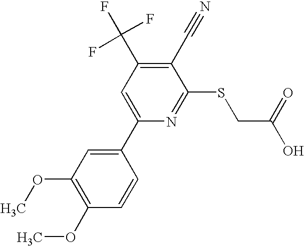 |
| 23 | 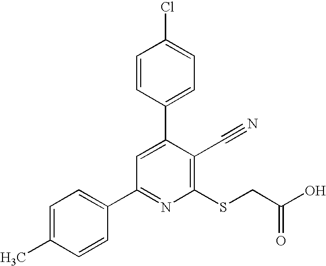 |
| 24 | 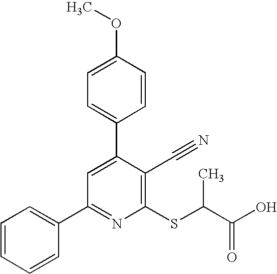 |
| 25 | 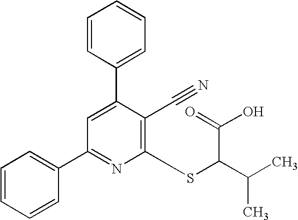 |
| 26 | 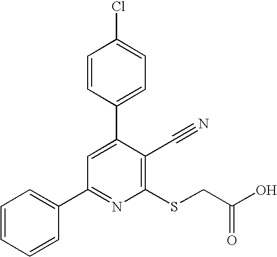 |
| 27 | 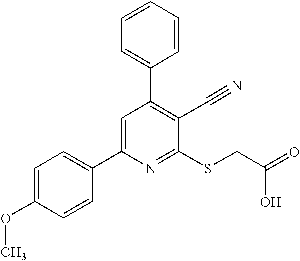 |
| 28 | 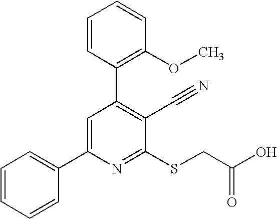 |
| 29 | 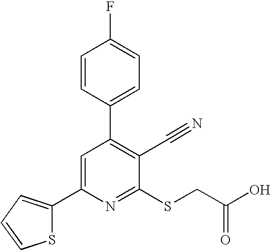 |
| 30 | 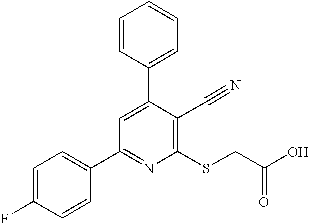 |
| 31 | 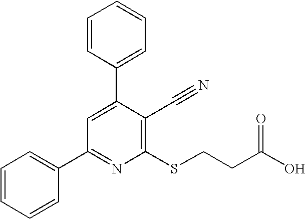 |
| 32 | 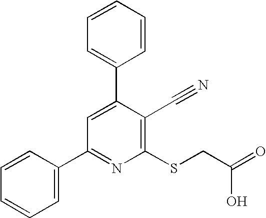 |
| 33 | 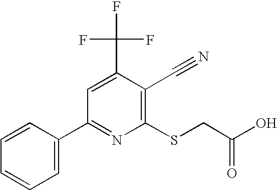 |
| 34 | 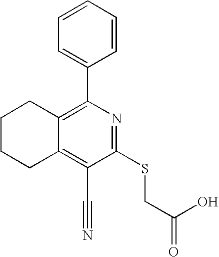 |
| 35 | 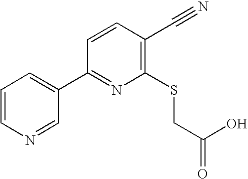 |

Various compounds from table 4 were subjected to the above glyoxalase I binding assay at a concentration of 20 μM (20 micromolar) as described in example 5. The following compounds exhibited inhibition, at 20 μM, of 30% or above: 1, 2, 3, 4, 9, 11, 13, 14, 25.

### Example 8

### HL60 Assay of 2-(2-Biphenyl-4-yl-2-oxo-ethylsulfanyl)-benzoic acid (compound 3)

The ethyl ester of compound 3 in table 4 was obtained from commercial sources and was also tested using the HL60 assay as described in example 6. This compound exhibited a 72% inhibition of proliferation in HL60s and had an IC₅₀ value of 7.5 μM.

1. A method of treating a condition which can be alleviated by inhibition of glyoxalase I, which method comprises administering to a patient in need of treatment an effective amount of a compound of formula I, or a pharmaceutically acceptable salt thereof: 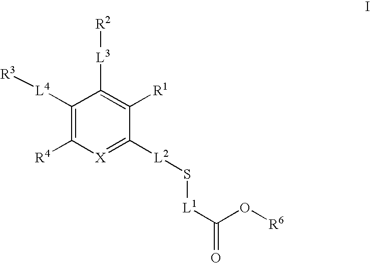
wherein
X is N or CH;
R¹ is H, cyano, halo, hydroxy, hydroxamic acid, sulfhydryl or —NH₂; or C₁₋₄ alkyl optionally substituted by cyano, halo, hydroxy, hydroxamic acid, sulfhydryl or —NH₂; or —OR, —NHR, —NR₂ or —SR wherein R is C₁₋₄ alkyl optionally substituted by cyano, halo, hydroxy, hydroxamic acid, sulfhydryl or —NH₂;
R² is H, CF₃; or optionally substituted C₅₋₆ aryl, C₃₋₇ cycloalkyl, C₅₋₇ heterocyclyl or together with R³ an optionally substituted C₃₋₄ alkylene group wherein L³ and L⁴ are single bonds thus forming a C₅₋₆ ring fused with the aromatic ring to which L³ and L⁴ are attached;
R³is H; or optionally substituted C₅₋₆ aryl, C₃₋₇ cycloalkyl, C₅₋₇ heterocyclyl or together with R² an optionally substituted C₃₋₄ alkylene group wherein L³ and L⁴ are single bonds thus forming a C₅₋₆ ring fused with the aromatic ring to which L³ and L⁴ are attached;
R⁴is H; or optionally substituted C₅₋₆ aryl or C₅₋₇ heterocyclyl;
R⁶ is selected from H or optionally substituted C₁₋₇ alkyl, C₅₋₆ aryl and C₁₋₄ alkylene-C₅₋₆ aryl;
L¹ is optionally substituted C₅₋₆ arylene, C₁₋₄ alkylene-C₅₋₆ arylene or -L⁵N(R⁵)L⁶-, or C₁₋₄ alkylene substituted by either C₁₋₇ alkyl or C₅₋₇ aryl, wherein L⁵ and L⁶ are independently selected from optionally substituted C₁₋₄ alkylene and C₅₋₆ arylene, and R⁵ is H or C₁₋₄ alkyl; and further wherein L¹ may be unsubstituted C₁₋₄ alkylene when X is N;
L² is a single bond; or optionally substituted C₁₋₄ alkylene or -L⁷C(═O)L⁸-, wherein L⁷ and L⁸ are independently selected from optionally substituted C₁₋₄ alkylene and a single bond; and
L³ and L⁴ are independently selected from a single bond, optionally substituted C₁₋₄ alkylene, -L⁹YN(OH)C(═O)L¹⁰- and -L⁹C(═O)N(OH)YL¹⁰-, wherein L⁹ and L¹⁰ are independently selected from optionally substituted C₁₋₄ alkylene, C₅₋₆ arylene, C₁₋₄ alkylene-C₅₋₆ arylene and a single bond, wherein Y is NH or a single bond.

2. A compound according to claim 1 wherein R¹ is chosen from the group consisting of H and cyano.

3. A compound according to claim 1 wherein R⁶ is H or C₁₋₇ alkyl.

4. A compound according to claim 1 wherein L¹ is chosen from the group consisting of phenylene, —CH(Ph)—, —CH₂-phenylene- and —CH₂C(═O)NH-phenylene-.

5. A compound according to claim 1 wherein L² is a single bond or —C(═O)CH₂—.

6. A compound according to claim 1 wherein L³ is chosen from the group consisting of a single bond, -L⁹YN(OH)C(═O)L¹⁰- and -L⁹C(═O)N(OH)YL¹⁰-, wherein L⁹ and L¹⁰ are independently selected from optionally substituted C₁₋₄ alkylene, C₅₋₆ arylene, C₁₋₄ alkylene-C₅₋₆ arylene and a single bond, and wherein Y is NH or a single bond.

7. A compound according to claim 6 wherein L³ is a single bond.

8. A compound according to claim 1 wherein L⁴ is chosen from the group consisting of a single bond, -L⁹YN(OH)C(═O)L¹⁰- and -L⁹C(═O)N(OH)YL¹⁰-, wherein L⁹ and L¹⁰ are independently selected from optionally substituted C₁₋₄ alkylene, C₅₋₆ arylene, C₁₄ alkylene-C₅₋₆ arylene and a single bond, and wherein Y is NH or a single bond.

9. A compound according to claim 8 wherein L⁴ is selected from the group consisting of —CH₂N(OH)C(═O)—, -phenylene-CH₂N(OH)C(═O)—, -phenylene-NHN(OH)C(═O)— and —CH₂C(═O)N(OH)—.

10. A compound according to claim 1 wherein X is CH.

11. A compound according to claim 10 wherein one of R¹, R² and R⁴ are H.

12. A compound according to claim 10 wherein two of R¹, R² and R⁴ are H.

13. A compound according to claim 10 wherein R¹, R² and R⁴ are all H.

14. A compound according to claim 10 wherein one of R² and R³ is optionally substituted C₅₋₆ aryl, C₃₋₇ cycloalkyl or C₅₋₇ heterocyclyl.

15. A compound according to claim 14 wherein R³ is optionally substituted C₅₋₆ aryl, C₃₋₇ cycloalkyl or C₅₋₇ heterocyclyl.

16. A compound according to claim 14 wherein R³ is optionally substituted phenyl or C₃₋₇ cycloalkyl.

17. A compound according to claim 14 wherein R³ is phenyl or cyclopentyl.

18. A compound according to claim 10 wherein L¹ is phenylene or —CH(Ph)—.

19. A compound according to claim 10 wherein one of L³ and L⁴ is a single bond.

20. A compound according to claim 19 wherein L³ is a single bond.

21. A compound according to claim 1 wherein X is N.

22. A compound according to claim 21 wherein R⁴ is selected from optionally substituted C₅₋₆ aryl and C₅₋₇ heterocyclyl.

23. A compound according to claim 21 wherein R¹ is cyano or hydroxamic acid.

24. A compound according to claim 21 wherein R² is selected from the group consisting of optionally substituted C₅₋₆ aryl, C₅₋₇ heterocyclyl, CF₃ and, together with R³, an optionally substituted butylene group wherein L³ and L⁴ are single bonds thus forming a C₆ ring fused with the aromatic ring to which L³ and L⁴ are attached.

25. A compound according to claim 24 wherein R² is selected from optionally substituted C₅₋₆ aryl or C₅₋₇ heterocyclyl.

26. A compound according to claim 24 wherein R² is selected from optionally substituted phenyl or thiophenyl.

27. A compound according to claim 24 wherein R² is selected from the group consisting of thiophenyl, phenyl, p-chlorophenyl, p-methoxyphenyl, o-methoxyphenyl and p-fluorophenyl.

28. A compound according to claim 24 wherein R² is a monosubstituted phenyl group with the substituent group being in the para position.

29. A compound according to claim 21 wherein R³ is H or, together with R², an optionally substituted butylene group wherein L³ and L⁴ are single bonds thus forming a C₆ ring fused with the aromatic ring to which L³ and L⁴ are attached.

30. A compound according to claim 29 wherein R³ is H and L⁴ is a single bond such that the compound is of formula Ib:

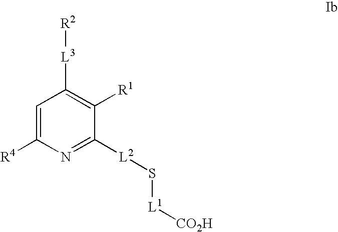

31. A pharmaceutical composition comprising a compound according to claim 1 or a pharmaceutically acceptable salt thereof together with a pharmaceutically acceptable carrier or diluent.

32.-33. (canceled)

34. A compound of formula I: 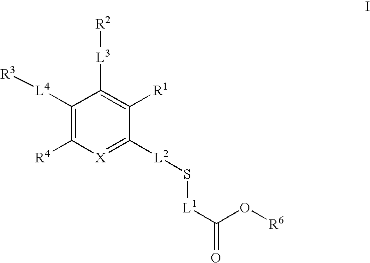
or a salt, solvate or chemically protected form thereof wherein
X is N or CH;
R¹ is H, cyano, halo, hydroxy, hydroxamic acid, sulfhydryl or —NH₂; or C₁₋₄ alkyl optionally substituted by cyano, halo, hydroxy, hydroxamic acid, sulfhydryl or —NH₂; or —OR, —NHR, —NR₂ or —SR wherein R is C₁₋₄ alkyl optionally substituted by cyano, halo, hydroxy, hydroxamic acid, sulfhydryl or —NH₂;
R² is H, CF₃; or optionally substituted C₅₋₆ aryl, C₃₋₇ cycloalkyl, C₅₋₇ heterocyclyl or together with R³ an optionally substituted C₃₋₄ alkylene group wherein L³ and L⁴ are single bonds thus forming a C₅₋₆ ring fused with the aromatic ring to which L³ and L⁴ are attached;
R³ is H; or optionally substituted C₅₋₆ aryl, C₃₋₇ cycloalkyl, C₅₋₇ heterocyclyl or together with R² an optionally substituted C₃₋₄ alkylene group wherein L³ and L⁴ are single bonds thus forming a C₅₋₆ ring fused with the aromatic ring to which L³ and L⁴ are attached;
R⁴ is H; or optionally substituted C₅₋₆ aryl or C₅₋₇ heterocyclyl;
R⁶ is selected from H or optionally substituted C₁₋₇ alkyl, C₅₋₆ aryl and C₁₋₄ alkylene-C₅₋₆ aryl;
L¹ is optionally substituted C₁₋₄ alkylene, C₅₋₆ arylene, C₁₋₄ alkylene-C₅₋₆ arylene or -L⁵N(R⁵)L⁶-, wherein L⁵ and L⁶ are independently selected from optionally substituted C₁₋₄ alkylene and C₅₋₆ arylene, and R⁵ is H or C₁₋₄ alkyl;
L² is a single bond; or optionally substituted C₁₋₄ alkylene or -L⁷C(═O)L⁸-, wherein L⁷ and L⁸ are independently selected from optionally substituted C₁₋₄ alkylene and a single bond; and
L³ and L⁴ are independently selected from a single bond, optionally substituted C₁₋₄ alkylene, -L⁹YN(OH)C(═O)L¹⁰- and -L⁹C(═O)N(OH)YL¹⁰-, wherein L⁹ and L¹⁰ are independently selected from optionally substituted C₁₋₄ alkylene, C₅₋₆ arylene, C₁₋₄ alkylene-C₅₋₆ arylene and a single bond, wherein Y is NH or a single bond; and wherein the compound contains at least one —C(═O)N(OH)— group.

35. A compound according to claim 34 wherein at least one of R¹, L³ or L⁴ comprises a —C(═O)N(OH)— group.

36. A compound according to claim 34 wherein L⁴ comprises a —C(═O)N(OH)— group.

37. A compound according to claim 34 wherein L⁴ is a L⁹-C(═O)N(OH)— group.

38. A compound according to claim 37 wherein L⁹ is selected from C₁₋₄ alkylene and C₅₋₆ arylene.

39. A compound according to claim 37 wherein L⁹ is methylene or phenylene.

40. A compound according to claim 34 wherein X is CH.

41. A compound according to claim 34 wherein at least one of R¹, R² and R⁴ is H.

42. A compound according to claim 34 wherein at least two of R¹, R² and R⁴ are H.

43. A compound according to claim 34 wherein all of R¹, R² and R⁴ are H.

44. A compound according to claim 34 wherein R³ is optionally substituted C₅₋₆ aryl.

45. A compound according to claim 44 wherein R³ is phenyl.

46. A compound according to claim 34 wherein R⁶ is H or C₁₋₇ alkyl.

47. A compound according to claim 46 wherein R⁶ is H or C₁₋₃ alkyl.

48. A compound according to claim 34 wherein L¹ is phenylene, —CH(Ph)—, —CH₂-phenylene- or —CH₂C(═O)NH-phenylene-.

49. A compound according to claim 34 wherein L² is a single bond or —C(═O)CH₂—.

50. A compound according to claim 34 wherein L³is a single bond.
